(19) United States
(12) Patent Application Publication
(10) Pub. No.: US 2023/0183180 A1
(43) Pub. Date: Jun. 15, 2023

(54) PROCESS OF MAKING ROXADUSTAT

(71) Applicant: SUZHOU PENGXU PHARMATECH CO., LTD., Suzhou, Jiangsu (CN)

(72) Inventors: Peng WANG, Forest Hill, NY (US); Pixu LI, Suzhou, Jiangsu (CN); Hailong YANG, Suzhou, Jiangsu (CN); Qiang WEI, Suzhou, Jiangsu (CN)

(73) Assignee: SUZHOU PENGXU PHARMATECH CO., LTD., Suzhou, Jiangsu (CN)

(21) Appl. No.: 17/924,850

(22) PCT Filed: Jun. 4, 2021
(86) PCT No.: PCT/US2021/036011

§ 371 Date: Nov. 11, 2022

(30) Foreign Application Priority Data
Jun. 13, 2020 (CN) 202010538639.0

(51) Int. Cl. C07D 217/26; B01J 25/02; B01J 21/02

(52) U.S. Cl. CPC C07D 217/26; B01J 25/02; B01J 21/02

(57) ABSTRACT

A process of making Roxadustat of the following formula:

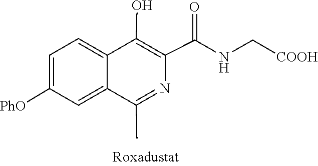 [SMILES: Cc1nc(C(=O)NCC(=O)O)c(O)c2ccc(Oc3ccccc3)cc12]

comprising converting a compound of formula VI:

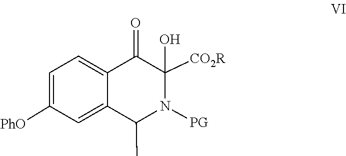

to Roxadustat, wherein R is a C₁-C₂₀ alkyl group, and PG is a protective group.

### CROSS REFERENCE TO RELATED APPLICATION

This application claims the benefit of China Patent Application No. 202010538639.0 filed on Jun. 13, 2020, the disclosure of which is incorporated herein by reference in its entirety.

### BACKGROUND OF THE INVENTION

### 1. Field of the Invention

The present invention relates to process for preparing Roxadustat and related intermediates.

### 2. Description of the Related Art

Renal anemia is one of the main complications of chronic kidney disease (CKD). With the development of CKD, the prevalence and severity of CKD related anemia increased gradually. The patient had severe fatigue and low quality of life. At present, the standard treatment of renal anemia is replacement of erythropoietin (EPO).

The drug reversibly binds to and inhibits HIF-prolyl hydroxylase enzymes that are responsible for the degradation of transcription factors in the HIF family under normal oxygen conditions. Inhibition of these enzymes reduces HIF breakdown and promotes HIF activity, leading to an increase in endogenous erythropoietin production, thereby enhancing erythropoiesis. It also reduces the expression of the peptide hormone hepcidin, improves iron availability and increases hemoglobin levels. HIF regulates the expression of genes in response to reduced oxygen levels, including genes required for erythropoiesis and iron metabolism. Roxadustat is approved in China and is under regulatory review in Japan for the treatment of anemia in patients with dialysis-dependent CKD. Studies are underway to investigate long-term cardiovascular outcomes with Roxadustat versus placebo (for non-dialysis-dependent CKD) or standard of care (for dialysis-dependent CKD).

Roxadustat (Ai Rui Zhuo® in China) is developed by FibroGen, in collaboration with Astellas and AstraZeneca. Roxadustat is the first in a new class of medicines, HIF-PH inhibitors that promote erythropoiesis, or red blood cell production, through increased endogenous production of erythropoietin; improved iron absorption and mobilization; and downregulation of hepcidin. Roxadustat is also in clinical development for anemia associated with myelodysplastic syndromes (MDS) and for chemotherapy-induced anemia (CIA). Roxadustat is approved in China, Japan, and Chile for the treatment of anemia of CKD in adult patients on dialysis (DD) and not on dialysis (NDD).

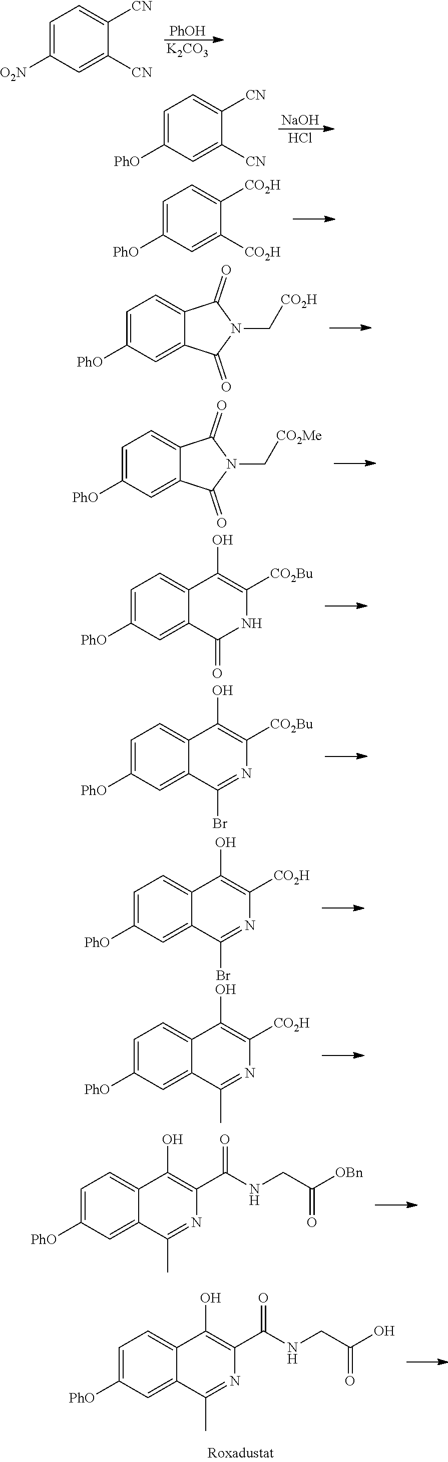

The preparation method was disclosed in compound patents U.S. Pat. No. 8,765,956B2 by Fibrogen. 3,4-Dicyano-nitrobenzene was used as the starting material to construct isoquinoline core structure through ring closing and rearrangement reactions. Methyl group was introduced through halogenation and transition metal-catalyzed cross coupling reaction. The glycine side chain was introduced followed by hydrolysis to furnish the synthesis of roxadustat. This route has some shortcomings, such as the poor selectivity of ring formation reaction, the use of expensive transition metal catalyst for the introduction of methyl, and the synthesis route is long.

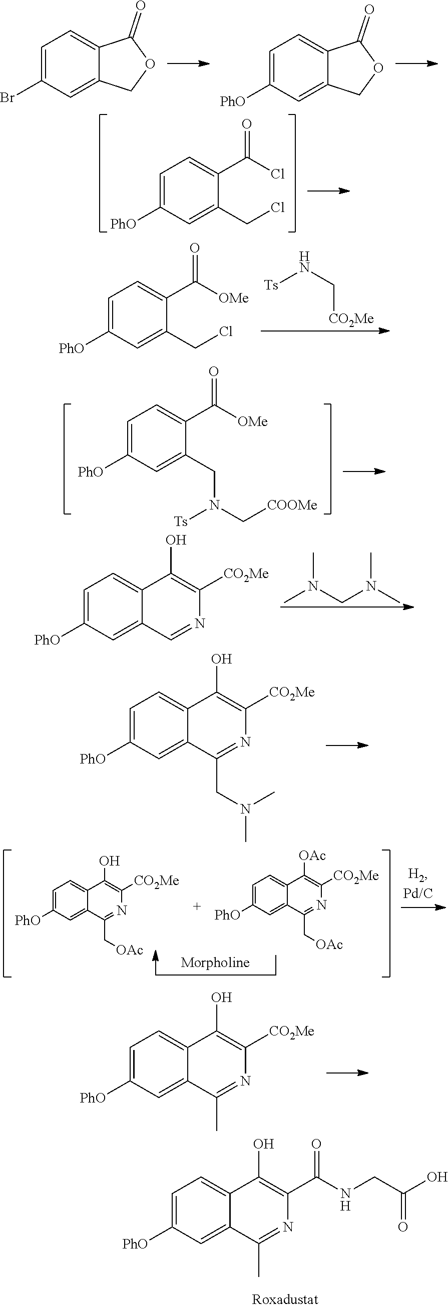

Later, a new preparation method was disclosed in process patents U.S. Pat. No. 9,340,511B. The route uses 5-bromophthalide as the starting material, introduces methyl glycine ester through ring opening reaction, constructs isoquinoline ring through ring closing reaction, and then introduces methyl group through amine reduction, protection and deprotection reaction. The whole step is long, and the deprotection reaction uses palladium on carbon.

After the investigation of current synthesis routes of Roxadustat, one of the characteristics of the existing strategy is that the methyl group is introduced through halogenation and coupling reaction after the isoquinoline ring is constructed. For example, the patent application WO2013013609A1 of Zhejiang bettapharma, the patent applications WO2018072662A1 and WO2019174631A1 of Shanghai Pharmaceutical Group, the patent application CN107602466 and CN108383787 etc. These processes introducing methyl group with transition metal-catalyzed cross coupling reaction are not ideal for commercial manufacturing of roxadustat due to high cost.

In addition, the introduction of methyl group on isoquinoline ring at earlier stage has also been reported. The key intermediate of methyl group substituted isoquinoline was synthesized from 5-bromo-3-methyl-phthalide via ring opening and rearrangement reaction. There are also some shortcomings in this route: the starting materials are not easily available and need to be synthesized in three steps. Patent application CN106478504 reported a synthetic method of isoquinoline intermediate through intramolecular high temperature cyclization reaction, but the reaction temperature was 200° C.

In patent application CN104892509B, tyrosine was used as the starting material. Isoquinoline ring was constructed through etherification and cyclization, and hydroxyl group was introduced in later stage. The whole route is straightforward and efficient. However, the etherification reaction has low selectivity, and the reaction of introducing hydroxyl group needs hydrogen peroxide, which is of potential safety issue, so it is good for scaling-up in commercial scale.

In view of the existing technical defects, it is of great practical significance to develop a simple, efficient, economical, and environmentally benign technology suitable for industrial production.

### SUMMARY OF THE INVENTION

One purpose of the present application is to provide a preparation of compound VI:

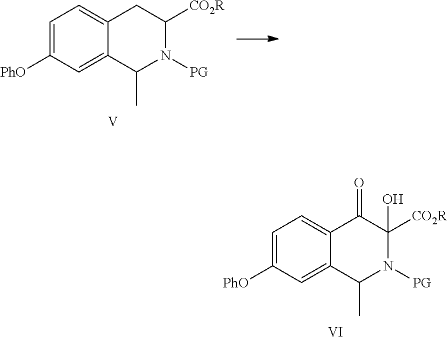

R=C₁-C₂₀ alkyl group; PG=protective group

In a specific embodiment, the reaction conditions for each reaction step are detailed below:

Synthesis of the compound of formula VI from the compound of formula V, the PG is selected from benzyloxycarbonyl, p-toluenesulfonyl, benzenesulfonyl, acetyl or propoxycarbonyl group.

The compound of formula V reacts under oxidation condition to prepare the compound of formula VI, the oxidant is ceric ammonium nitrate.

A further purpose of the present application is to provide a method to prepare the compound VII:

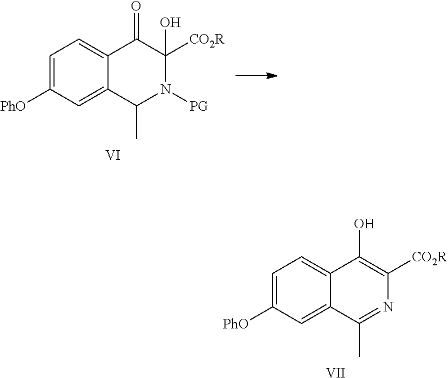

R=C₁-C₂₀ alkyl group; PG=protective group.

In a specific embodiment, the reaction conditions for each reaction step are detailed below:

Synthesis of the compound of formula VII from the compound of formula VI, the PG is selected from benzyloxycarbonyl, p-toluenesulfonyl, benzenesulfonyl, acetyl or propoxycarbonyl group.

The compound of formula VI reacts under acidic conditions to prepare the compound of formula VII. The acid is selected from hydrochloric acid, hydrobromic acid, sulfuric acid, nitric acid, acetic acid, formic acid.

A further purpose of the present application is to provide a method to prepare the compound IV:

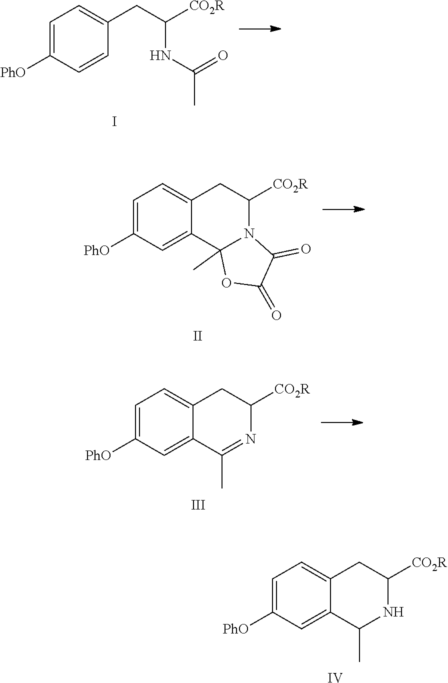

R=C₁-C₂₀ alkyl group.

In a specific embodiment, the reaction conditions for each reaction step are detailed below:

Synthesis of the compound of formula IV from the compound of formula I:

(1) Compound I reacts with oxalyl chloride and ferric chloride to give compound II;

(2) Compound II is deprotected under acidic conditions to give compound III;

(3) Compound III is reduced to give compound IV.

The acid in step (2) is selected from hydrochloric acid, hydrobromic acid, sulfuric acid, nitric acid, acetic acid, formic acid and mixtures thereof. The reduction reaction conditions of the step (3) are selected from sodium borohydride, lithium borohydride, palladium carbon/hydrogen or Raney Ni/hydrogen.

A further purpose of the present application is to provide a method to prepare the compound VII:

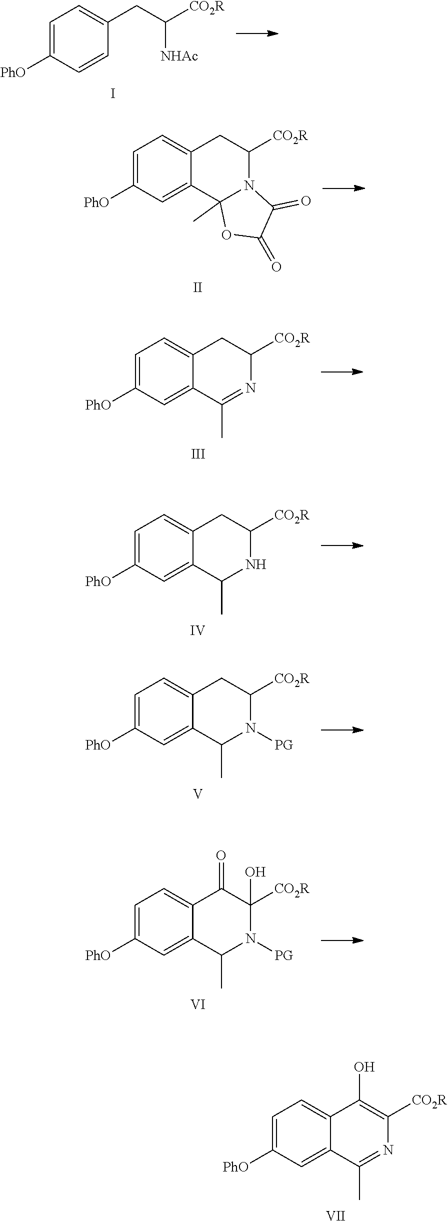

R=C₁-C₂₀ alkyl group; PG=protective group.

In a specific embodiment, the reaction conditions for each reaction step are detailed below:

Synthesis of the compound of formula VII from the compound of formula VI, the PG is selected from benzyloxycarbonyl, p-toluenesulfonyl, benzenesulfonyl, acetyl or propoxycarbonyl group.

Synthesis of the compound of formula VII from the compound of formula I:

(1) Compound I reacts with oxalyl chloride and ferric chloride to give compound II;

(2) Compound II is deprotected under acidic conditions to give compound III;

(3) Compound III is reduced to give compound IV;

(4) The amino group in compound IV is protected to obtain compound V;

(5) The compound of formula V reacts under oxidation conditions to prepare the compound of formula VI;

(6) The compound of formula VI reacts under acidic conditions to prepare the compound of formula VII.

The acid in step (2) is selected from hydrochloric acid, hydrobromic acid, sulfuric acid, nitric acid, acetic acid, formic acid and mixtures thereof. The reductant of the step (3) is selected from the group consisting of sodium borohydride, lithium borohydride, palladium carbon/hydrogen, Raney Ni/hydrogen, and combinations thereof. The protecting group of the step (4) is selected from the group consisting of benzyloxycarbonyl, p-toluenesulfonyl, benzenesulfonyl, acetyl, propoxycarbonyl, and combinations thereof. The oxidant in step (5) is ceric ammonium nitrate. The acid in step (6) is selected from the group consisting of hydrochloric acid, hydrobromic acid, sulfuric acid, nitric acid, acetic acid, formic acid, and mixtures thereof.

A further purpose of the present application is to provide an intermediate compound II for the synthesis of Roxadustat.

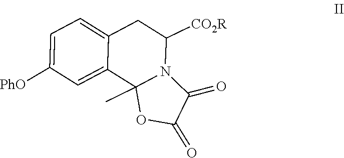

R=C₁-C₂₀ alkyl group; The compound of formula II is a single configuration or mixture of (5S,10bR), (5R,10bR), (5S,10bS), (5R,10bS).

A further purpose of the present application is to provide an intermediate compound III or its salt for the synthesis of Roxadustat.

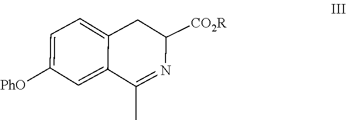

R=C₁-C₂₀ alkyl group; The compound of formula III is a single configuration or mixture of (3S), (3R).

A further purpose of the present application is to provide an intermediate compound V for the synthesis of roxadustat.

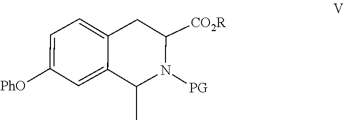

The compound of formula II is a single configuration or mixture of (1S,3S), (1R,3S), (1S,3R), (1R,3R), R=C₁-C₂₀ alkyl group; The PG is selected from benzyloxycarbonyl, p-toluenesulfonyl, benzenesulfonyl, acetyl or propoxycarbonyl group.

A further purpose of the present application is to provide an intermediate compound VI for the synthesis of Roxadustat:

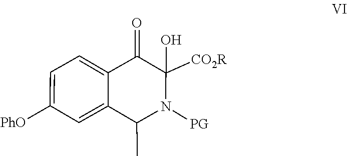

The compound of formula VI is a single configuration or mixture of (1S,3S), (1R,3S), (1S,3R), (1R,3R), R=C₁-C₂₀ alkyl group; The PG is selected from benzyloxycarbonyl, p-toluenesulfonyl, benzenesulfonyl, acetyl or propoxycarbonyl group.

Compared with the prior art, the present method has the following advantages:

1. Simple and efficient introduction of methyl group;
2. Mild reaction conditions, suitable for scale-up production.

### DESCRIPTION OF PREFERRED EMBODIMENTS

### Examples

### Example 1: Synthesis of Compound 6

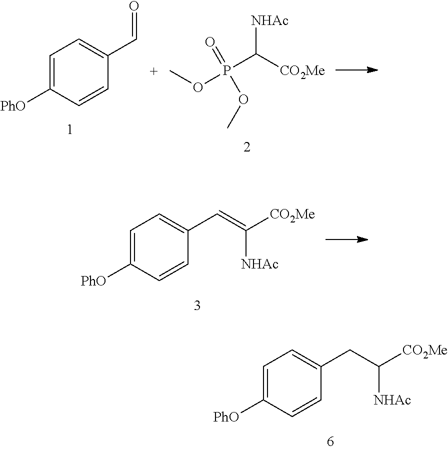

To a round-bottom flask were added compound 1 (134.0 g), compound 2 (212.3 g) and DCM (675 mL). DBU (133.8 g) was added dropwise at 5-15° C. After 2 h, the reaction mixture was quenched with H₂O. Acetic acid was added to adjust pH=6˜7. The layers were separated. The aqueous phase was extracted with DCM twice. The organic phases were combined and concentrated. The mixture was added MTBE and stirred at room temperature for 1 h. The suspension was filtered to give compound 3 as an off-white solid (199.2 g, 94.6% yield, 96.0% purity).

¹H NMR (400 MHz, CDCl₃) δ 7.47-7.35 (m, 2H), 7.40-7.34 (m, 4H), 7.22-7.13 (m, 1H), 7.06-7.02 (m, 2H), 6.98-6.92 (m, 2H), 3.82 (s, 3H), 2.12 (s, 3H). Mass: [M+H]⁺: 312.3;

To a reactor were added MeOH (1 L), compound 3 (100 g) and catalyst Rh (Rc, Sp-Duanphos)(NBD)BF₄ (1 g). The reactor was degassed and refilled with H₂ (2.0 MPa) three times. The mixture was stirred at room temperature under H₂ (3.0˜4.0 MPa) for overnight. The reaction mixture was filtered. The filtrate was concentrated to dryness. The crude product was recrystallized with EtOAc/heptane (150 mL/200 mL) to give compound 6 (92.0 g, 91.5% yield, 100% purity).

¹H NMR (400 MHz, DMSO-d6) δ 8.38-8.36 (m, 1H), 7.41-7.36 (m, 2H), 7.25-7.21 (m, 2H), 7.15-7.11 (m, 1H), 6.99-6.93 (m, 4H), 4.47-4.41 (m, 1H), 3.60 (s, 3H), 3.02-2.84 (m, 2H), 1.81 (s, 3H).

Mass: [M+H]⁺: 314.3.

### Example 2: Synthesis of Compound 8

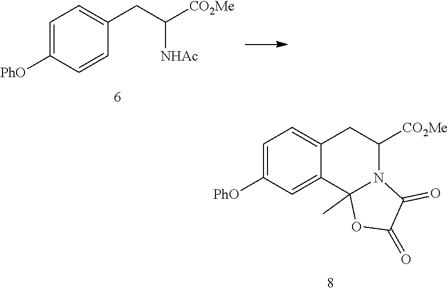 [SMILES: COC(=O)C(Cc1ccc(Oc2ccccc2)cc1)NC(C)=O ; COC(=O)C1Cc2ccc(Oc3ccccc3)cc2C2(C)OC(=O)C(=O)N12]

To a round-bottom flask were added compound 6 (2.8 g) and DCM (28 mL). (COCl)₂ (1.3 g) was added dropwise at 20˜30° C. The mixture was stirred for 2 h. FeCl₃ (1.7 g) was added in portions. The mixture was stirred overnight at room temperature. The reaction mixture was quenched with H₂O. The layers were separated. The organic phase was concentrated to dryness. The crude product was purified by silica gel column chromatography eluting with EA/heptane=1/3 to give compound 8 as a light yellow solid (2.4 g, 62.5% yield, 97.0% purity).

¹H NMR (400 MHz, DMSO-d6) δ 7.44-7.38 (m, 3H), 7.20-7.15 (m, 1H), 7.09-7.08 (m, 1H), 7.06-7.01 (m, 3H), 4.91-4.87 (m, 1H), 3.79 (s, 3H), 3.53-3.47 (m, 1H), 3.30-3.24 (m, 1H), 1.87 (s, 3H).

Mass: [M+H]⁺: 368.3.

### Example 3: Synthesis of Compound 9

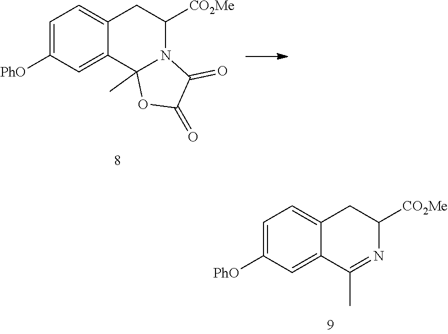 [SMILES: COC(=O)C1Cc2ccc(Oc3ccccc3)cc2C2(C)OC(=O)C(=O)N12 ; COC(=O)C1Cc2ccc(Oc3ccccc3)cc2C(C)=N1]

To a round-bottom flask were added compound 8 (1.4 g), EtOH (14 mL) and H₂SO₄. The mixture was heated and refluxed at 75˜85° C. overnight. The mixture was concentrated, quenched with saturated sodium bicarbonate solution, and extracted with EtOAc (20 mL) twice. The combined organic phase was concentrated to dryness. The crude product was purified by silica gel column chromatography eluting with EA/heptane=1/6 to give compound 9 as a light yellow solid (0.9 g, 80% yield, 97.3% purity).

¹H NMR (400 MHz, CDCl₃) δ 7.38-7.34 (m, 2H), 7.21-7.18 (m, 2H), 7.13 (s, 1H), 7.05-7.00 (m, 3H), 4.26-4.20 (m, 1H), 3.84 (s, 3H), 2.98-2.92 (m, 2H), 2.41 (s, 3H).

Mass: [M+H]⁺: 296.3.

### Example 4: Synthesis of Compound 10

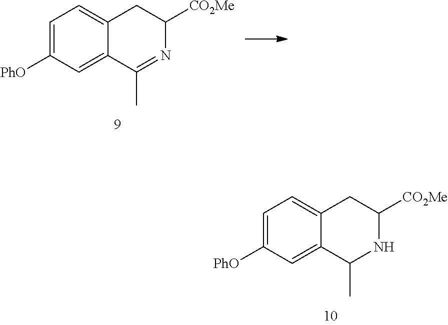 [SMILES: COC(=O)C1Cc2ccc(Oc3ccccc3)cc2C(C)=N1 ; COC(=O)C1Cc2ccc(Oc3ccccc3)cc2C(C)N1]

To a 250 mL reactor were added compound 9, Pd/C (or Raney Ni) and IPAC. The reactor was degassed and refilled with H₂ three times. The mixture was stirred at room temperature under H₂ (4.0˜5.0 MPa) for 3 h. The reaction mixture was filtered, and the filtrate was concentrated to dryness. The crude product was purified by silica gel column chromatography eluting with EtOAc/heptane=1/6 to give compound 10 as a light yellow oil (970 mg, 60.0% yield, 98.5% purity).

Mass: [M+H]⁺: 298.3.

### Example 5: Synthesis of Compound 12

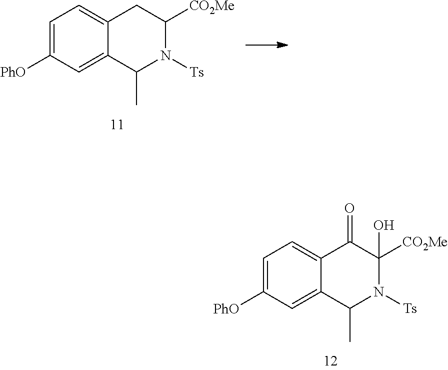 [SMILES: COC(=O)C1Cc2ccc(Oc3ccccc3)cc2C(C)N1S(=O)(=O)c1ccc(C)cc1 ; COC(=O)C1(O)C(=O)c2ccc(Oc3ccccc3)cc2C(C)N1S(=O)(=O)c1ccc(C)cc1]

To a round-bottom flask were added compound 11 (0.6 g), MeCN (6 mL) and H₂O (6 mL). CAN (3.65 g) was added in portions and the reaction mixture was stirred for 3 h. After completion of the reaction, EtOAc (30 mL) was added, and the layers were separated. The organic phase was concentrated to dryness. The crude product was purified by silica gel column chromatography eluting with EtOAc/heptane=1/8 to give compound 12 as a light yellow solid (150 mg, 21% yield).

Mass: [M−H₂O+H]⁺: 464.2.

### Example 6: Synthesis of Compound 13

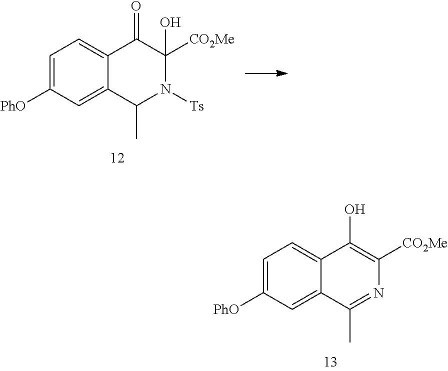 [SMILES: COC(=O)C1(O)C(=O)c2ccc(Oc3ccccc3)cc2C(C)N1S(=O)(=O)c1ccc(C)cc1 ; COC(=O)c1nc(C)c2cc(Oc3ccccc3)ccc2c1O]

To a round-bottom flask were added compound 12 (0.13 g), 33% HBr—AcOH solution (1 mL) and phenol (26 mg). The reaction mixture was stirred for 3 h. EtOAc (20 mL) was added, and the layers were separated. The organic phase was washed with 20% potassium carbonate aqueous solution. The organic phase was concentrated to dryness. The crude product was purified by silica gel column chromatography eluting with EtOAc/heptane=1/5 to give compound 13 as a light-yellow solid (10 mg, 11% yield, 98.0% purity).

¹H NMR (400 MHz, CDCl₃) δ 11.71 (s, 1H), 8.42-8.40 (m, 1H), 7.52-7.42 (m, 4H), 7.28-7.23 (m, 1H), 7.16-7.13 (m, 2H), 4.10 (s, 3H), 2.77 (s, 3H).

Mass: [M+H]⁺: 310.1.

### Example 7: Synthesis of Compound 7

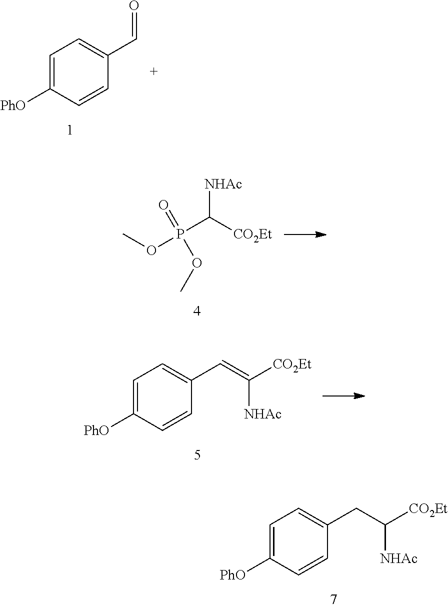

To a round-bottom flask were added compound 1 (45.0 g), compound 4 (60.4 g) and DCM (225 mL). DBU (44.4 g) was added dropwise at 0˜10° C. The reaction mixture was warmed to room temperature and stirred for 1 h. The reaction mixture was quenched with H₂O (225 mL). The layers were separated. The organic phase was concentrated to dryness. The crude product was added MTBE (180 mL) and stirred at room temperature for 1 h. The suspension was filtered to give compound 5 as a white solid (566.8 g, 90.4% yield, 100% purity).

¹H NMR (400 MHz, CDCl₃) δ 7.48-7.37 (m, 4H), 7.20-7.13 (m, 2H), 7.09-7.06 (m, 2H), 6.98-6.96 (m, 2H), 4.31 (q, J=7.1 Hz, 2H), 2.16 (s, 3H), 1.37 (t, J=7.1 Hz, 3H).

Mass: [M+H]⁺: 326.3.

To a 2 L reactor were added EtOH (1 L), compound 5 (65 g) and catalyst Rh (Rc,Sp-Duanphos) (NBD)BF₄ (0.5 g). The reactor was degassed and refilled with H₂ (2.0 MPa) three times. The mixture was stirred at 40˜50° C. under H₂ (1.8 MPa) overnight. The reaction mixture was filtered. The filtrate was concentrated to dryness. The crude product was recrystallized with EtOAc/heptane to give compound 7 as an off-white solid (59.5 g, 91.0% yield, 100% purity).

1H NMR (400 MHz, DMSO-d6) δ 8.37-8.35 (m, 1H), 7.40-7.36 (m, 2H), 7.25-7.23 (m, 2H), 7.14-7.11 (m, 1H), 6.99-6.92 (m, 4H), 4.46-4.40 (m, 1H), 4.07-4.02 (m, 2H), 3.00-2.86 (m, 2H), 1.82 (s, 3H), 1.12 (t, J=7.1 Hz, 3H).

Mass: [M+H]⁺: 328.3.

### Example 8: Synthesis of Compound 15

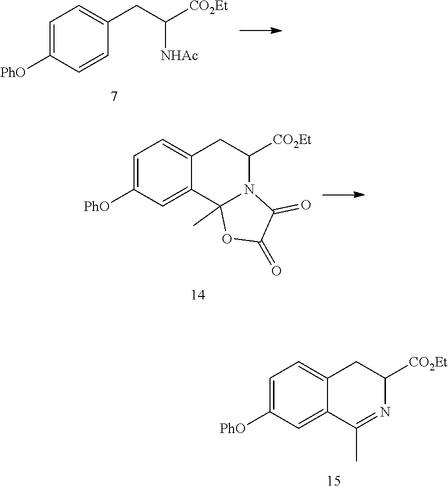 [SMILES: CCOC(=O)C(Cc1ccc(Oc2ccccc2)cc1)NC(C)=O ; CCOC(=O)C1Cc2ccc(Oc3ccccc3)cc2C2(C)OC(=O)C(=O)N12 ; CCOC(=O)C1Cc2ccc(Oc3ccccc3)cc2C(C)=N1]

To a round-bottom flask were added compound 7 (160 g) and DCM (1.28 L). (COCl)₂ (69.0 g) was added dropwise at room temperature. The mixture was stirred for 2 h. FeCl₃ (96.1 g) was added in portions at −15° C.˜−10° C. The mixture was warmed to room temperature and stirred for 2 h. H₂O (800 mL) and DCM (1.6 L) were added, and the layers were separated. One eighth of the organic phase was concentrated to dryness. The crude product was added EtOH (200 mL) and stirred for 3 h. The suspension was filtered to give compound 14 (8.1 g, 34.8% yield, 95.5% purity). Seven eighths of the organic phase was concentrated to dryness and added EtOH (1.26 L) and sulfuric acid (0.14 L). The mixture was heated to 70˜80° C. and stirred overnight. The mixture was quenched with saturated sodium bicarbonate solution. The pH of the mixture was adjusted to 6˜7. The layers were separated. The organic layer was concentrated to dryness. The crude product was purified by silica gel column chromatography eluting with EA/heptane=1/20 to give compound 15 as a yellow oil (110 g, 83.0% yield, 98.0% purity).

### Compound 14:

Mass: [M+H]⁺: 382.2;

### Compound 15:

¹H NMR (400 MHz, CDCl₃) δ 7.37-7.33 (m, 2H), 7.20-7.17 (m, 2H), 7.14-7.11 (m, 1H), 7.04-6.99 (m, 3H), 4.29 (q, J=7.1 Hz, 2H), 4.26-4.19 (m, 1H), 3.03-2.86 (m, 2H), 2.40 (d, J=2.0 Hz, 3H), 1.32 (t, J=7.1 Hz, 3H).

Compound 15 Mass: [M+H]⁺: 310.2.

### Example 9: Synthesis of Compound 16

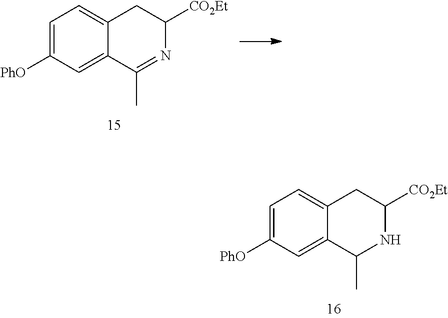 [SMILES: CCOC(=O)C1Cc2ccc(Oc3ccccc3)cc2C(C)=N1 ; CCOC(=O)C1Cc2ccc(Oc3ccccc3)cc2C(C)N1]

To a 250 mL reactor were compound 15 (15 g), 10% Pd/C (1.5 g) and EtOH (120 mL). The reactor was degassed and refilled with H₂ three times. The mixture was stirred at room temperature under H₂ (5.0 MPa) for 2 h. The mixture was filtered. The filtrate was concentrated to dryness. The crude product 16 was a yellow oil liquid (13.8 g, 91.3% yield, 95.4% purity).

¹H NMR (400 MHz, CDCl₃) δ 7.33-7.29 (m, 2H), 7.09-7.05 (m, 2H), 6.99-6.96 (m, 2H), 6.88-6.87 (m 1H), 6.82-6.79 (m, 1H), 4.28-4.22 (m, 2H), 4.13-4.11 m, 1H), 3.74-3.70 (m, 1H), 3.07-2.92 (m, 2H), 2.02 (s, 1H), 1.46 (d, J=6.5 Hz, 3H), 1.32 (t, J=7.1 Hz, 3H).

Mass: [M+H]⁺: 312.3.

### Example 10: Synthesis of Compound 17

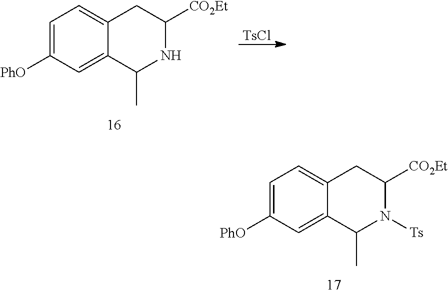

To a round-bottom flask were added compound 17 (22.4 g), DCM (150 mL), TEA (10.2 g) and tosyl chloride (19.24 g). The mixture was stirred at 40˜50° C. overnight. The reaction mixture was quenched with H₂O (100 mL). The layers were separated. The organic phase was concentrated to dryness. The crude product was purified by silica gel column chromatography eluting with EtOAc/heptane=1/8 to give compound 17 as a white solid (25.1 g, 74.1% yield, 99.35% purity).

¹H NMR (400 MHz, CDCl₃) δ 7.67-7.65 (m, 2H), 7.35-7.31 (m, 2H), 7.21-7.19 (m, 2H), 7.13-7.09 (m, 1H), 7.03-7.01 (m, 1H), 6.93-6.90 (m, 2H), 6.78-6.75 (m, 1H), 6.65-6.64 (m, 1H), 4.93 (q, J=7.0 Hz, 1H), 4.56-4.53 (m, 1H), 4.23 (q, J=7.1 Hz, 2H), 3.01-2.96 (m, 2H), 2.38 (s, 3H), 1.48 (d, J=7.0 Hz, 3H), 1.29 (t, J=7.1 Hz, 3H).

Mass: [M+H]⁺: 466.3.

### Example 11: Synthesis of Compound 18

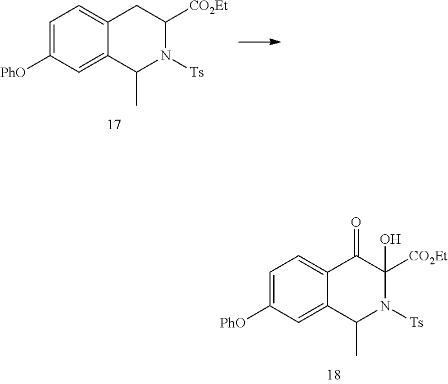 [SMILES: CCOC(=O)C1Cc2ccc(Oc3ccccc3)cc2C(C)N1S(=O)(=O)c1ccc(C)cc1 ; CCOC(=O)C1(O)C(=O)c2ccc(Oc3ccccc3)cc2C(C)N1S(=O)(=O)c1ccc(C)cc1]

To a round-bottom flask were added compound 17 (1.6 g), MeCN (16 mL) and H₂O (16 mL). CAN (9.44 g) was added in portions. The reaction mixture was stirred for 3 h. EtOAc (100 mL) was added. The layers were separated. The organic phase was concentrated to dryness. The crude product was purified by silica gel column chromatography eluting with EtOAc/heptane=1/8 to give compound 18 as a light-yellow solid (0.95 g, 58% yield).

¹H NMR (400 MHz, CDCl₃) δ 7.94-7.92 (m, 3H), 7.48-7.44 (m, 2H), 7.34-7.26 (m, 3H), 7.13-7.10 (m, 2H), 6.99-6.96 (m, 1H), 6.71-6.70 (m, 1H), 4.86 (q, J=6.8 Hz, 1H), 4.35-4.29 (m, 2H), 2.46 (s, 3H), 1.27 (d, J=6.8 Hz, 3H), 1.24 (t, J=6.8 Hz, 3H).

Mass: [M−H₂O+H]⁺: 478.2.

### Example 12: Synthesis of Compound 30

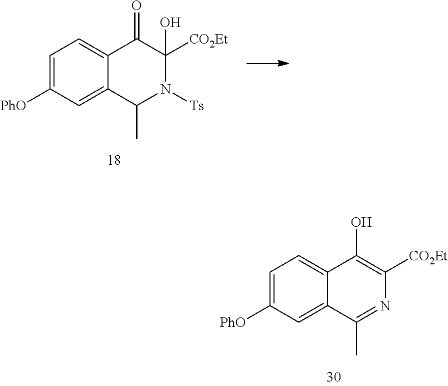 [SMILES: CCOC(=O)C1(O)C(=O)c2ccc(Oc3ccccc3)cc2C(C)N1S(=O)(=O)c1ccc(C)cc1 ; CCOC(=O)c1nc(C)c2cc(Oc3ccccc3)ccc2c1O]

To a round-bottom flask were added compound 18 (0.2 g), 33% HBr—AcOH solution (2 mL) and phenol (39.4 mg). The mixture was stirred at room temperature. After completion of the reaction, EtOAc (20 mL) was added. The layers were separated. The organic layer was washed with 20% potassium carbonate aqueous solution. The organic phase was concentrated to dryness. The crude product was purified by silica gel column chromatography eluting with EtOAc/heptane=1/5 to give compound 30 as a light yellow solid (90 mg, 67% yield, 95.0% purity).

¹H NMR (400 MHz, DMSO-d6) δ 11.61 (s, 1H), 8.31-8.29 (m, 1H), 7.57-7.56 (m, 1H), 7.53-7.47 (m, 3H), 7.29-7.25 (s, 1H), 7.20-7.18 (m, 2H), 4.45 (d, J=7.1 Hz, 2H), 2.63 (s, 3H), 1.39 (t, J=7.1 Hz, 3H).

Mass: [M+H]⁺: 324.1.

### Example 13: Synthesis of Compound 19

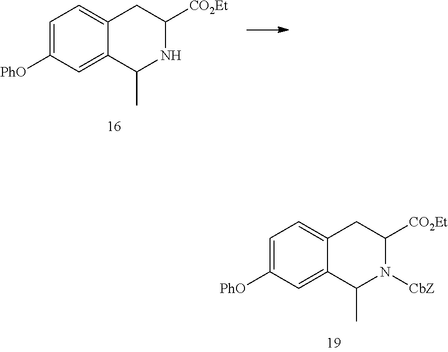

To a round-bottom flask were added THF (80 mL), H₂O (80 mL), compound 16 (8.01 g) and potassium bicarbonate (3.9 g). The mixture was cooled to 0˜10° C. CbzCl (5.33 g) was added in portions. The mixture was stirred at room temperature for 1 h. The layers were separated. The organic phase was dried with anhydrous sodium sulfate. After filtration and concentration, the crude product was purified by silica gel column chromatography eluting with EtOAc/heptane=1/15 to give compound 19 as an oil (7.7 g, 70% yield, 99.05% purity).

¹H NMR (400 MHz, CDCl₃) δ 7.40-7.34 (m, 7H), 7.17-7.11 (m, 2H), 7.03-7.01 (m, 2H), 6.89-6.81 (m, 2H), 5.33-5.10 (m, 3H), 4.74-4.22 (m, 2H), 4.07-4.04 (m, 1H), 3.19-3.12 (m, 2H), 1.52 (d, J=6.9 Hz, 3H), 1.32-1.15 (m, 3H). Mass: [M+H]+: 446.3.

### Example 14: Synthesis of Compound 20

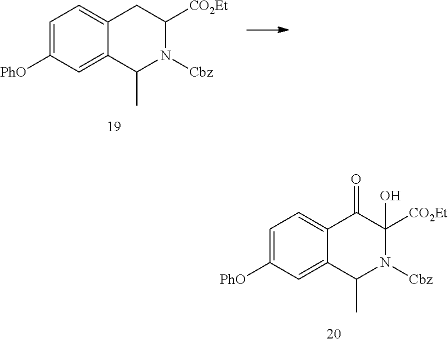 [SMILES: CCOC(=O)C1Cc2ccc(Oc3ccccc3)cc2C(C)N1C(=O)OCc1ccccc1 ; CCOC(=O)C1(O)C(=O)c2ccc(Oc3ccccc3)cc2C(C)N1C(=O)OCc1ccccc1]

To a round-bottom flask were added compound 19 (6 g), MeCN (60 mL) and H₂O (60 mL). CAN (36.9 g) was added in portions. The reaction mixture was stirred for 3 h. EtOAc (200 mL) was added. The layers were separated. The organic phase was concentrated to dryness. The crude product was purified by silica gel column chromatography eluting with EtOAc/heptane=1/5 to give compound 20 as a light yellow solid (3.65 g, 56% yield, 95.0% purity).

¹H NMR (400 MHz, CDCl₃) δ 7.94-7.78 (m, 1H), 7.51-7.46 (m, 2H), 7.41-7.27 (m, 7H), 7.24-7.15 (m, 3H), 7.02-6.97 (m, 1H), 5.47-5.46 (m, 1H), 5.27-5.19 (m, 2H), 4.06-4.01 (m, 2H), 1.49-1.34 (m, 3H), 1.10-1.06 (m, 3H).

Mass: [M−H₂O+H]⁺: 458.3.

### Example 15: Synthesis of Compound 30

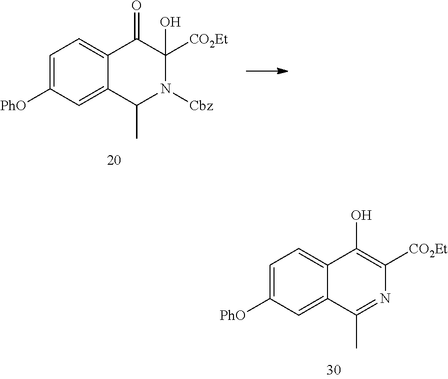 [SMILES: CCOC(=O)C1(O)C(=O)c2ccc(Oc3ccccc3)cc2C(C)N1C(=O)OCc1ccccc1 ; CCOC(=O)c1nc(C)c2cc(Oc3ccccc3)ccc2c1O]

To a round-bottom flask were added compound 18 (3.5 g), acetic acid (12 mL) and 33% HBr—AcOH solution (12 mL). The mixture was stirred at room temperature. After completion of the reaction, EtOAc (100 mL) was added and the layers were separated. The organic phase was washed with 20% potassium carbonate aqueous solution. The organic phase was concentrated to dryness. The crude product was purified by silica gel column chromatography eluting with EtOAc/heptane=1/5 to give compound 30 as a light yellow solid (2.05 g, 86% yield, 99.5% purity).

¹H NMR (400 MHz, CDCl₃) δ 11.61 (s, 1H), 8.31-8.29 (m, 1H), 7.57-7.56 (m, 1H), 7.53-7.47 (m, 3H), 7.29-7.25 (s, 1H), 7.20-7.18 (m, 2H), 4.45 (d, J=7.1 Hz, 2H), 2.63 (s, 3H), 1.39 (t, J=7.1 Hz, 3H).

Mass: [M+H]⁺: 324.1.

### Example 16: Synthesis of Compound 21

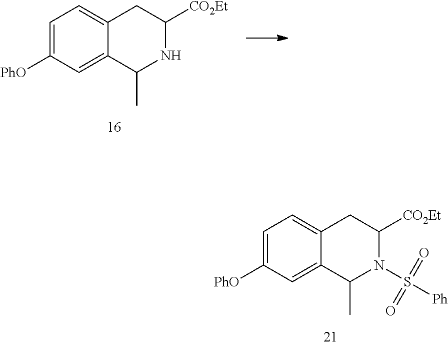 [SMILES: CCOC(=O)C1Cc2ccc(Oc3ccccc3)cc2C(C)N1 ; CCOC(=O)C1Cc2ccc(Oc3ccccc3)cc2C(C)N1S(=O)(=O)c1ccccc1]

To a round-bottom flask were added compound 16 (8.0 g), DCM (80 mL), TEA (5.2 g) and p-TsCl (6.9 g). The mixture was stirred at 40° C. overnight. The mixture was quenched with H₂O (80 mL). The layers were separated. The organic phase was concentrated to dryness. The crude product was purified by silica gel column chromatography eluting with EtOAc/heptane=1/9 to give compound 21 as an oil (8.9 g, 77.3% yield, 100% purity).

¹H NMR (400 MHz, CDCl₃) δ 7.79-7.77 (m, 2H), 7.53-7.48 (m, 1H), 7.42-7.40 (m, 2H), 7.33-7.31 (m, 2H), 7.11-7.10 (m, 1H), 7.02-7.00 (m, 1H), 6.91-6.89 (m, 2H), 6.78-6.75 (m, 1H), 6.64-6.63 (m, 1H), 4.97-4.92 (m, 1H), 4.57-4.53 (m, 1H), 4.26-4.20 (m, 2H), 3.16-2.97 (m, 2H), 1.50-1.47 (m, 3H), 1.31-1.26 (m, 3H).

Mass: [M+H]⁺: 452.7.

### Example 17: Synthesis of Compound 22

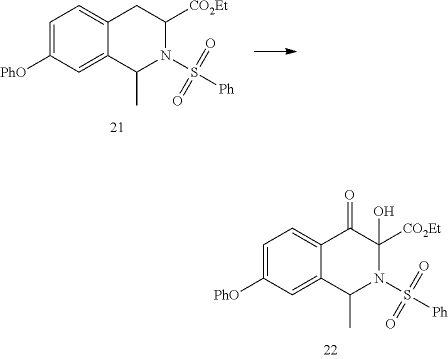 [SMILES: CCOC(=O)C1Cc2ccc(Oc3ccccc3)cc2C(C)N1S(=O)(=O)c1ccccc1 ; CCOC(=O)C1(O)C(=O)c2ccc(Oc3ccccc3)cc2C(C)N1S(=O)(=O)c1ccccc1]

To a round-bottom flask were added compound 21 (6 g), MeCN (60 mL) and H₂O (60 mL). CAN (36.9 g) was added in portions. The reaction mixture was stirred for 3 h. EtOAc (60 mL) was added. The layers were separated. The organic phase was concentrated to dryness. The crude product was purified by silica gel column chromatography eluting with EtOAc/heptane=1/8 to give compound 22 as a light yellow solid (2.9 g, 47% yield, 93.0% purity).

Mass: [M−H₂O+H]⁺: 464.3.

### Example 18: Synthesis of Compound 30

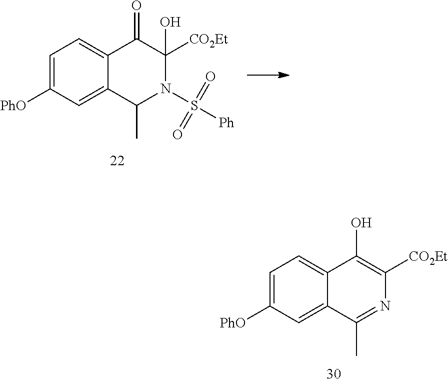 [SMILES: CCOC(=O)C1(O)C(=O)c2ccc(Oc3ccccc3)cc2C(C)N1S(=O)(=O)c1ccccc1 ; CCOC(=O)c1nc(C)c2cc(Oc3ccccc3)ccc2c1O]

To a round-bottom flask were added compound 22 (2.9 g), 33% HBr—AcOH solution (12 mL) and phenol (0.59 g). The mixture was stirred at room temperature. After completion of the reaction, EtOAc (30 mL) was added. The layers were separated. The organic phase was washed with 20% potassium carbonate aqueous solution. The organic phase was concentrated to dryness. The crude product was purified by silica gel column chromatography eluting with EtOAc/heptane=1/5 to give compound 30 as a light yellow solid (1.51 g, 74% yield, 96.2% purity).

¹H NMR (400 MHz, CDCl₃) δ 11.61 (s, 1H), 8.31-8.29 (m, 1H), 7.57-7.56 (m, 1H), 7.53-7.47 (m, 3H), 7.29-7.25 (s, 1H), 7.20-7.18 (m, 2H), 4.45 (d, J=7.1 Hz, 2H), 2.63 (s, 3H), 1.39 (t, J=7.1 Hz, 3H).

Mass: [M+H]⁺: 324.1.

### Example 19: Synthesis of Compound 23

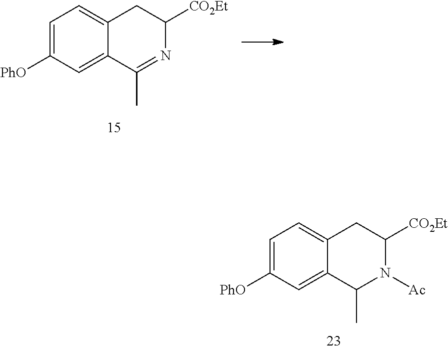 [SMILES: CCOC(=O)C1Cc2ccc(Oc3ccccc3)cc2C(C)=N1 ; CCOC(=O)C1Cc2ccc(Oc3ccccc3)cc2C(C)N1C(C)=O]

To a reactor were compound 15 (6.0 g), EtOH (60 mL), acetic anhydride (3.96 g) and 10% Pd/C (0.6 g). The reactor was degassed and refilled with H₂ three times. The mixture was stirred at room temperature under H₂ (3.0˜4.0 MPa). After completion of the reaction, the mixture was filtered. The filtrate was concentrated to dryness. The crude product was purified by silica gel column chromatography eluting with EtOAc/heptane=1/6 to give compound 23 as a white solid (4.2 g, 57.0% yield, purity 97.5%).

¹H NMR (400 MHz, CDCl₃) δ 7.39-7.34 (m, 2H), 7.19-7.14 (m, 2H), 7.04-7.01 (m, 2H), 6.91-6.82 (m, 2H), 5.64-4.22 (m, 2H), 4.26-4.22 (m, 2H), 3.17-3.14 (m, 2H), 2.26-2.17 (m, 3H), 1.58-1.42 (m, 3H), 1.44-1.31 (m, 3H).

Mass: [M+H]⁺: 354.3.

### Example 20: Synthesis of Compound 24

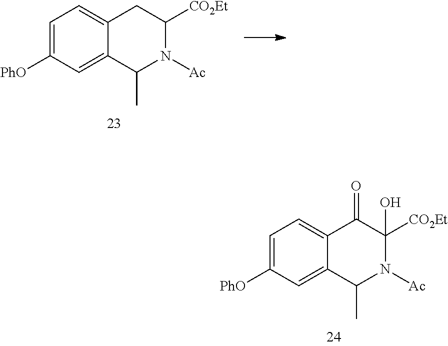 [SMILES: CCOC(=O)C1Cc2ccc(Oc3ccccc3)cc2C(C)N1C(C)=O ; CCOC(=O)C1(O)C(=O)c2ccc(Oc3ccccc3)cc2C(C)N1C(C)=O]

To a round-bottom flask were added compound 23 (1 g), MeCN (10 mL) and H₂O (10 mL). CAN (7.8 g) was added in portions. The reaction mixture was stirred at room temperature for 3 h. EtOAc (30 mL) was added. The layers were separated. The organic phase was concentrated to dryness. The crude product was purified by silica gel column chromatography eluting with EtOAc/heptane=1/5 to give compound 24 as a light yellow solid (0.6 g, 55% yield, 98.0% purity).

¹H NMR (400 MHz, CDCl₃) δ 7.99-7.97 (m, 1H), 7.47-7.43 (m, 2H), 7.27-7.25 (m, 1H), 7.13-7.11 (m, 2H), 7.00-6.98 (m, 1H), 6.81-6.80 (m, 1H), 5.20 (s, 1H), 5.05-5.04 (m, 1H), 4.22-4.19 (m, 2H), 5.64-4.22 (m, 2H), 4.26-4.22 (m, 2H), 3.17-3.14 (m, 2H), 2.26-2.17 (m, 3H), 1.58-1.42 (m, 3H), 1.44-1.31 (m, 3H).

Mass: [M+H]⁺: 384.6.

### Example 21: Synthesis of Compound 30

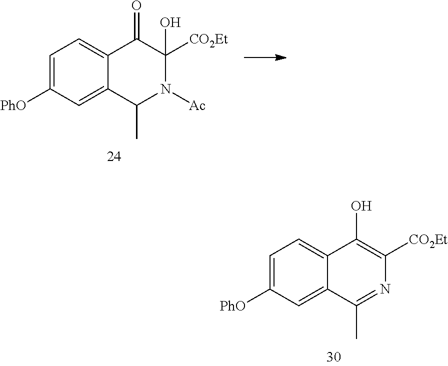 [SMILES: CCOC(=O)C1(O)C(=O)c2ccc(Oc3ccccc3)cc2C(C)N1C(C)=O ; CCOC(=O)c1nc(C)c2cc(Oc3ccccc3)ccc2c1O]

To a round-bottom flask were compound 24 (30 mg), THF (2 mL) and p-toluene sulfonic acid (15 mg). The mixture was stirred and heated to 60˜70° C. The target product was detected by HPLC and LCMS.

Mass: [M+H]⁺: 324.1.

### Example 22: Synthesis of Compound 25

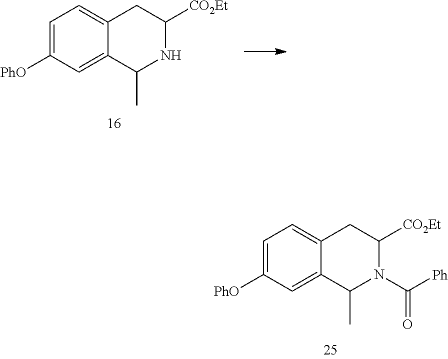 [SMILES: CCOC(=O)C1Cc2ccc(Oc3ccccc3)cc2C(C)N1 ; CCOC(=O)C1Cc2ccc(Oc3ccccc3)cc2C(C)N1C(=O)c1ccccc1]

To a round-bottom flask were added compound 16 (12 g), DCM (120 mL), TEA (5.8 g) and benzoyl chloride (8.2 g). The mixture was stirred at room temperature. After completion of the reaction, the mixture was quenched with H₂O (30 mL). The layers were separated. The organic phase was concentrated to dryness. The crude product was purified by silica gel column chromatography eluting with EtOAc/heptane=1/5 to give compound 25 as a white solid (14.5 g, 89.7% yield, 97.5% purity).

¹H NMR (400 MHz, DMSO-d6) δ 7.48-7.22 (m, 8H), 7.13-6.78 (m, 5H), 5.54-4.72 (m, 2H), 4.24-3.98 (m, 2H), 3.38-3.32 (m, 1H), 3.19-3.10 (m, 1H), 1.48-1.35 (m, 3H), 1.29-1.08 (m, 3H).

Mass: [M+H]⁺: 416.3.

### Example 23: Synthesis of Compound 26

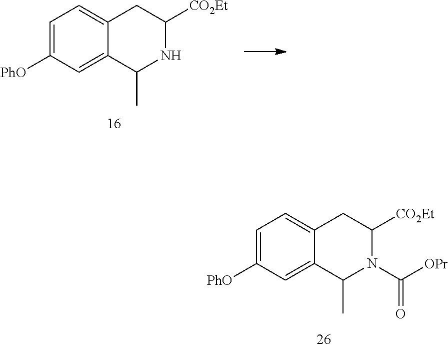

To a round-bottom flask were added compound 16 (10 g), THF (150 mL), H₂O (150 mL), sodium bicarbonate (4.0 g), and propyl chlorocarbonate (4.7 g). The mixture was stirred at room temperature. After completion of the reaction, EtOAc (200 mL) was added. The layers were separated. The organic phase was concentrated to dryness. The crude product was purified by silica gel column chromatography eluting with EtOAc/heptane=1/8 to give compound 26 as a light yellow oil (9.9 g, 77.0% yield, 99.8% purity).

¹H NMR (400 MHz, CDCl₃) δ 7.38-7.33 (m, 2H), 7.16-7.10 (m, 2H), 7.03-7.00 (m, 2H), 6.89-6.84 (m, 2H), 5.30-5.13 (m, 1H), 4.70-4.49 (m, 1H), 4.26-4.18 (m, 2H), 4.11-4.02 (m, 2H), 3.17-3.12 (m, 2H), 1.75-1.60 (m, 2H), 1.52-1.45 (m, 3H), 1.33-1.26 (m, 3H), 1.01-0.92 (m, 3H).

Mass: [M+H]⁺: 398.3.

### Example 24: Synthesis of Compound 27

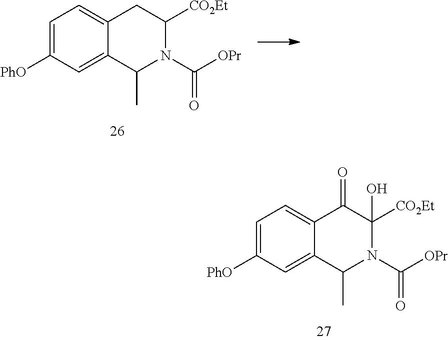

To a round-bottom flask were added compound 26 (6 g), MeCN (60 mL) and H₂O (60 mL). CAN (41.4 g) was added in portions. The reaction mixture was stirred for 3 h. EtOAc (100 mL) was added. The layers were separated. The organic phase was concentrated to dryness. The crude product was purified by silica gel column chromatography eluting with EtOAc/heptane=1/8 to give compound 27 as a light yellow solid (4.9 g, 96.3% yield, 75.0% purity).

¹H NMR (400 MHz, CDCl₃) δ 8.02-7.90 (m, 1H), 7.48-7.43 (m, 2H), 7.30-7.26 (m, 1H), 7.14-7.10 (m, 2H), 7.01-6.98 (m, 1H), 6.83-6.81 (m, 1H), 5.38-5.32 (m, 1H), 4.27-4.26 (m, 2H), 4.20-4.09 (m, 2H), 1.76-1.66 (m, 2H), 1.57-1.54 (m, 3H), 1.23-1.19 (m, 2H), 1.01-0.97 (m, 3H).

Mass: [M−H₂O+H]⁺: 410.3.

### Example 25: Synthesis of Roxadustat

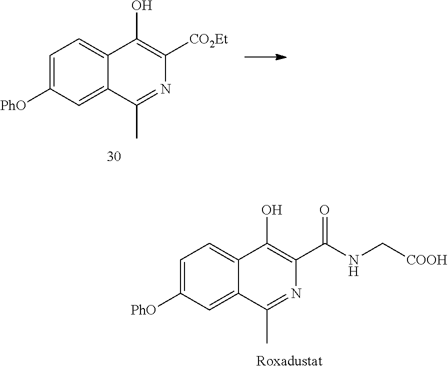 [SMILES: CCOC(=O)c1nc(C)c2cc(Oc3ccccc3)ccc2c1O ; Cc1nc(C(=O)NCC(=O)O)c(O)c2ccc(Oc3ccccc3)cc12]

To a pressure tube were added compound 30 (2 g), glycine (1.4 g) and sodium methylate. The reaction mixture was heated to 110-120° C. for 4 h. The reaction was cooled to room temperature. The target compound roxadustat was confirmed by HPLC and LC-MS.

Compound Roxadustat: Mass: [M−H]⁻: 351.1.

1. A method of making Roxadustat of the following formula: 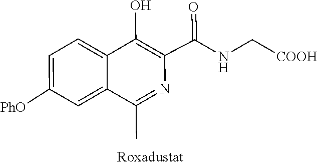 [SMILES: Cc1nc(C(=O)NCC(=O)O)c(O)c2ccc(Oc3ccccc3)cc12]
comprising converting a compound of formula VI: 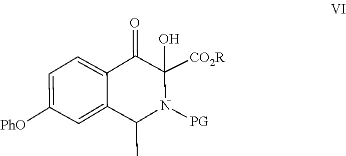
to Roxadustat, wherein R is a C₁-C₂₀ alkyl group, and PG is a protective group.

2. The method of claim 1 wherein the protecting group is selected from the group consisting of benzyloxycarbonyl, p-toluenesulfonyl, benzenesulfonyl, acetyl, and propoxycarbonyl groups.

3. The method of claim 1 wherein the step of converting the compound of formula VI to Roxadustat comprises converting the compound of formula VI to a compound of formula VII: 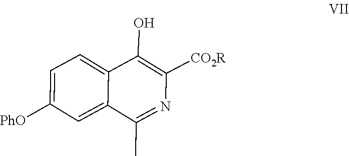
and then converting the compound of formula VII to Roxadustat.

4. The method of claim 3 wherein the step of converting the compound of formula VI to the compound of formula VII is conducted in the presence of at least one acid.

5. The method of claim 4 wherein the acid is selected from the group consisting of hydrochloric acid, hydrobromic acid, sulfuric acid, nitric acid, acetic acid, and formic acid.

6. The method of claim 3 wherein the step of converting the compound of formula VII to Roxadustat is conducted with glycine in the presence of a base.

7. The method of claim 6 wherein the base is selected from the group consisting of sodium ethylate, potassium ethylate, sodium tert-butoxide, potassium tert-butoxide, and combinations thereof.

8. The method of claim 1 comprising a step of converting a compound of formula V: 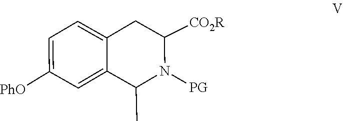
to the compound of formula VI.

9. The method of claim 8 wherein the step of converting the compound of formula V to the compound of formula VI is conducted under oxidative conditions.

10. The method of claim 9 wherein the step of converting the compound of formula V to the compound of formula VI is conducted in the presence of ceric ammonium nitrate as an oxidant.

11. The method of claim 8 comprising a step of converting a compound of formula IV: 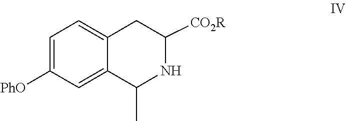
to the compound of formula V.

12. The method of claim 11 comprising making the compound of formula IV through the following synthetic scheme:

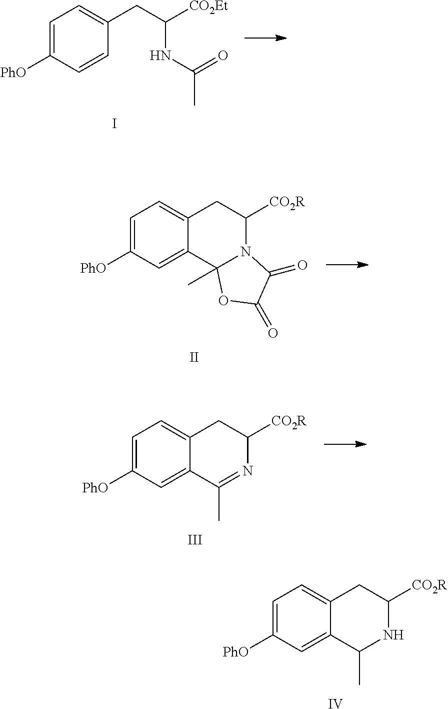

13. The method of claim 12 comprising reacting the compound of formula I with oxalyl chloride and ferric chloride to produce the compound of formula II.

14. The method of claim 12 comprising deprotecting the compound of formula II under acidic conditions to produce the compound of formula III.

15. The method of claim 14 wherein the deprotecting is conducted in the presence of an acid selected from the group consisting of hydrochloric acid, hydrobromic acid, sulfuric acid, nitric acid, acetic acid, formic acid, and combinations thereof.

16. The method of claim 12 comprising reducing the compound of formula III to produce the compound of formula IV.

17. The method of claim 16 wherein the reducing is conducted in the presence of a reducing agent selected from the group consisting of sodium borohydride, lithium borohydride, palladium carbon/hydrogen, Raney Ni/hydrogen, and combinations thereof.

18. A compound of formula VI 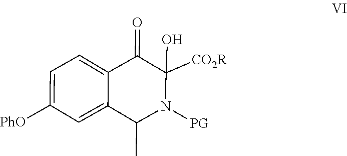
wherein R is a C₁-C₂₀ alkyl group, and PG is a protective group.

19. A process of making the compound of formula VI of claim 18 comprising converting a compound of formula V: 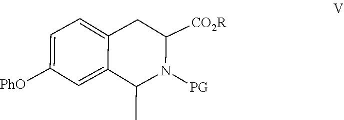
to the compound of formula VI.

20. The process of claim 19 wherein the step of converting the compound of formula V to the compound of formula VI is carried out in the presence of ceric ammonium nitrate as an oxidant.

21. A compound selected from the group consisting of formulae II, III, and V: 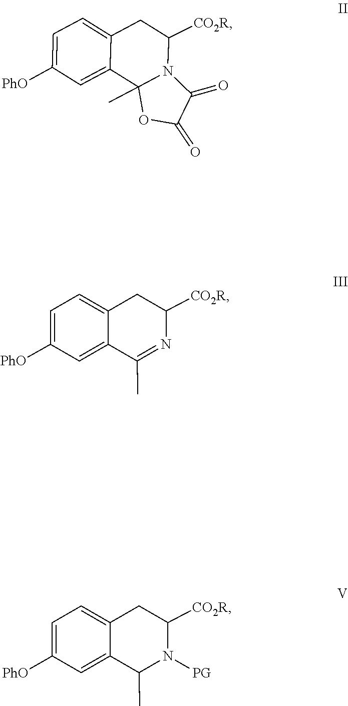
wherein R is a C₁-C₂₀ alkyl group, and PG is a protective group.
